(19) United States
(12) Patent Application Publication
(10) Pub. No.: US 2023/0149092 A1
(43) Pub. Date: May 18, 2023

(54) SYSTEMS AND METHODS FOR COMPENSATING FOR OBSTRUCTIONS IN MEDICAL IMAGES

(71) Applicant: Stryker Corporation, Kalamazoo, MI (US)

(72) Inventors: Brian FOUTS, Morgan Hill, CA (US); Cole Kincaid HUNTER, Santa Clara, CA (US); Joshua SUBRAHMANYAM, Aurora, CO (US)

(73) Assignee: Stryker Corporation, Kalamazoo, MI (US)

(21) Appl. No.: 18/056,237

(22) Filed: Nov. 16, 2022

Related U.S. Application Data
(60) Provisional application No. 63/264,171, filed on Nov. 16, 2021.

(51) Int. Cl. A61B 34/10; A61B 90/00

(52) U.S. Cl. CPC A61B 34/10; A61B 90/37

(57) ABSTRACT

A method for determining an attribute associated with anatomy of interest of a patient includes receiving first image data capturing the anatomy of interest of the patient and at least one obstruction obscuring at least a portion of the anatomy of interest of the patient; generating, using at least one machine learning model, second image data in which at least a portion of the obstruction is replaced; and determining at least one attribute associated with the anatomy of interest based on the second image data.

### CROSS-REFERENCE TO RELATED APPLICATIONS

This application claims the benefit of U.S. Provisional Application No. 63/264,171, filed Nov. 16, 2021, the entire contents of which are hereby incorporated by reference herein.

### FIELD

This disclosure relates to medical imaging in general and, more particularly, to medical imaging in support of minimally invasive surgical procedures.

### BACKGROUND

Orthopedics is a medical specialty that focuses on the diagnosis, correction, prevention, and treatment of patients with skeletal conditions, including for example conditions or disorders of the bones, joints, muscles, ligaments, tendons, nerves and skin, which make up the musculoskeletal system. Joint injuries or conditions such as those of the hip joint or other joints can occur from overuse or over-stretching or due to other factors, including genetic factors that may cause deviations from “normal” joint morphology.

The current trend in orthopedic surgery is to treat joint injuries or pathologies using minimally-invasive techniques such as joint arthroscopy in which an endoscope is inserted into the joint through a small incision. Procedures performed arthroscopically include debridement of bony pathologies in which portions of bone in a joint that deviate from a “normal” or target morphology are removed. During a debridement procedure, the surgeon uses an endoscopic camera to view the debridement area, but because the resulting endoscopic image has a limited field of view, the surgeon may not be able to view the entire pathology all at once.

X-ray imaging can be used to view a greater portion of the pathology than may be provided by endoscopic imaging. A C-arm X-ray machine may be used to generate X-ray imaging intraoperatively for display to a surgeon for viewing the pathology during the treatment procedure. Such intraoperative X-ray imaging may be generated while surgical instruments, such as debridement tools, remain in the surgical cavity. As a result, the surgical instruments may be captured in the X-ray imaging, often obscuring portions of the anatomy of interest.

### SUMMARY

According to various aspects, systems and methods include detecting an obstruction in medical imaging that obscures anatomy of interest, removing the obstruction from the imaging, and filling-in the portion of the imaging associated with the removed obstruction with a representation of the obscured portion of the anatomy using one or more machine learning models. The resulting obstruction-free imaging can be displayed to medical personnel for better visualization of the anatomy of interest. Additionally or alternatively, the obstruction-free imaging be analyzed to determine an attribute of the anatomy of interest. For example, where the obstruction obscures a portion of the anatomy that impacts image-based analysis of the anatomy, the image-based analysis may be enabled or improved by replacement of the obstruction with a representation of the anatomy.

According to an aspect, the obstruction detection and the obstruction replacement are performed using different machine learning models. Optionally, a first machine learning model segments the imaging, outputting a mask that indicates which pixels of the image belong to the obstruction and which are anatomical pixels. A second machine learning model may then in-paint the region associated with the obstruction, taking as an input the original imaging and the mask generated by the first machine learning model and output imaging where the obstruction is replaced by a representation of what may be obscured by the obstruction.

According to an aspect, a method for removing an obstruction from imaging of anatomy of a patient includes receiving first image data capturing anatomy of interest of the patient and at least one obstruction obscuring a portion of the anatomy of interest; detecting the at least one obstruction in the first image data using a first machine learning model; and generating, using a second machine learning model that is different than the first machine learning model, second image data in which at least a portion of the at least one obstruction is replaced based on the anatomy of interest.

Optionally, the at least one obstruction is replaced by a representation of the portion of the anatomy of interest obscured by the at least one obstruction.

Optionally, the first image data comprises X-ray image data.

Optionally, an output from the first machine learning model is an input to the second machine learning model.

Optionally, the first machine learning model outputs a mask that indicates which pixels of the first image data correspond to the at least one obstruction.

Optionally, the second machine learning model generates the representation based on the mask and the first image data. The mask may be enlarged prior to being provided as an input to the second machine learning model.

Optionally, the second image data is displayed intraoperatively.

Optionally, the second image data comprises a representation of the at least one obstruction. The representation may include at least one of a silhouette and an outline.

Optionally, the method further includes determining at least one attribute associated with the anatomy of interest based on the second image data, generating a visual guidance associated with the anatomy of interest based on the determined at least one attribute, and adding the visual guidance to the second image data.

Optionally, the visual guidance provides guidance for bone removal.

Optionally, the at least one obstruction comprises an instrument or an implant.

According to an aspect, a system for removing an obstruction from imaging of anatomy of a patient includes one or more processors, memory, and one or more programs stored in the memory for execution by the one or more processors and including instructions for: receiving first image data capturing anatomy of interest of the patient and at least one obstruction obscuring a portion of the anatomy of interest; detecting the at least one obstruction in the first image data using a first machine learning model; and generating, using a second machine learning model that is different than the first machine learning model, second image data in which at least a portion of the at least one obstruction is replaced based on the anatomy of interest.

Optionally, the at least one obstruction is replaced by a representation of the portion of the anatomy of interest obscured by the at least one obstruction.

Optionally, the first image data comprises X-ray image data.

Optionally, an output from the first machine learning model is an input to the second machine learning model.

Optionally, the first machine learning model outputs a mask that indicates which pixels of the first image data correspond to the at least one obstruction. The second machine learning model may generate the representation based on the mask and the first image data. The mask may be enlarged prior to being provided as an input to the second machine learning model.

Optionally, the second image data is displayed intraoperatively.

Optionally, the second image data comprises a representation of the at least one obstruction.

Optionally, the representation comprises at least one of a silhouette and an outline.

Optionally, the one or more programs include further instructions for determining at least one attribute associated with the anatomy of interest based on the second image data, generating a visual guidance associated with the anatomy of interest based on the determined at least one attribute, and adding the visual guidance to the second image data.

Optionally, the visual guidance provides guidance for bone removal.

Optionally, the at least one obstruction comprises an instrument or an implant.

According to an aspect, a method for determining an attribute associated with anatomy of interest of a patient includes receiving first image data capturing the anatomy of interest of the patient and at least one obstruction obscuring at least a portion of the anatomy of interest of the patient; generating, using at least one machine learning model, second image data in which at least a portion of the obstruction is replaced; and determining at least one attribute associated with the anatomy of interest based on the second image data.

Optionally, the method further includes generating a visual guidance associated with the anatomy of interest based on the determined at least one attribute and adding the visual guidance to the second image data.

Optionally, the visual guidance provides guidance for bone removal.

Optionally, the second image data is displayed intraoperatively for guiding a surgical procedure.

Optionally, determining the at least one attribute comprises identifying at least a portion of a perimeter of the anatomy of interest based at least in part on the representation of the at least a portion of the anatomy of interest obscured by the obstruction. The obstruction may obscure the at least a portion of the perimeter in the first image data.

Optionally, the first image data is an X-ray image.

Optionally, generating the second image data comprises using a first machine learning model to identify the obstruction and using a second machine learning model to generate the second image data based on the identification of the obstruction by the first machine learning model.

Optionally, the method further includes displaying the second image data with a representation of the at least one obstruction overlaid on the representation of the at least a portion of the anatomy of interest.

Optionally, the at least one obstruction is at least one surgical instrument.

According to an aspect, a system for determining an attribute associated with anatomy of interest of a patient includes one or more processors, memory, and one or more programs stored in the memory for execution by the one or more processors and including instructions for: receiving first image data capturing the anatomy of interest of the patient and at least one obstruction obscuring at least a portion of the anatomy of interest of the patient; generating, using at least one machine learning model, second image data in which at least a portion of the obstruction is replaced; and determining at least one attribute associated with the anatomy of interest based on the second image data.

Optionally, the one or more programs include further instructions for generating a visual guidance associated with the anatomy of interest based on the determined at least one attribute and adding the visual guidance to the second image data.

Optionally, the visual guidance provides guidance for bone removal. The second image data may be displayed intraoperatively for guiding a surgical procedure.

Optionally, determining the at least one attribute comprises identifying at least a portion of a perimeter of the anatomy of interest based at least in part on the representation of the at least a portion of the anatomy of interest obscured by the obstruction. The obstruction may obscure the at least a portion of the perimeter in the first image data.

Optionally, the first image data is an X-ray image.

Optionally, generating the second image data comprises using a first machine learning model to identify the obstruction and using a second machine learning model to generate the second image data based on the identification of the obstruction by the first machine learning model.

Optionally, the one or more programs include further instructions for displaying the second image data with a representation of the at least one obstruction overlaid on the representation of the at least a portion of the anatomy of interest.

Optionally, the at least one obstruction is at least one surgical instrument.

According to an aspect, a method for training a machine learning model to identify obstructions in medical images includes manually identifying at least one obstruction in at least one first training image; generating at least one artificial training image by adding at least a portion of the at least one obstruction extracted from the at least one first training image to at least one obstruction-free image; generating masks for the at least one obstruction in the at least one first training image and the at least one artificial training image; and training a machine learning model with the masks, the at least one first training image, and the at least one artificial training image.

Optionally, the at least one obstruction comprises a surgical instrument or an implant.

Optionally, the at least one first training image is an X-ray image.

Optionally, the at least on obstruction is outlined in the at least one first training image.

Optionally, the machine learning model is a convolutional neural network.

Optionally, multiple artificial training images are generated via different rotations and/or positions of the at least one obstruction.

According to an aspect, a system for training a machine learning model to identify obstructions in medical images includes one or more processors, memory, and one or more programs stored in the memory for execution by the one or more processors and including instructions for: receiving user input manually identifying at least one obstruction in at least one first training image; generating at least one artificial training image that includes the at least one obstruction extracted from the at least one first training image added to at least one obstruction-free image; generating masks for the at least one obstruction in the at least one first training image and the at least one artificial training image; and training a machine learning model with the masks, the at least one first training image, and the at least one artificial training image.

Optionally, the at least one obstruction comprises a surgical instrument or an implant.

Optionally, the at least one first training image is an X-ray image.

Optionally, the at least on obstruction is outlined in the at least one first training image.

Optionally, the machine learning model is a convolutional neural network.

Optionally, multiple artificial training images are generated via different rotations and/or positions of the at least one obstruction.

According to an aspect, a method for determining an attribute associated with anatomy of interest of a patient includes receiving first image data capturing the anatomy of interest of the patient and at least one obstruction obscuring at least a portion of the anatomy of interest of the patient; determining a location of the obstruction relative to the anatomy of interest within the first image data using at least one machine learning model; and determining at least one attribute associated with the anatomy of interest based on the location of the obstruction relative to the anatomy of interest.

Optionally, the method further comprises generating a visual guidance associated with the anatomy of interest based on the determined at least one attribute and displaying the visual guidance. The visual guidance may provide guidance for bone removal. The visual guidance may be displayed intraoperatively for guiding a surgical procedure.

Optionally, determining the at least one attribute comprises identifying at least a portion of a perimeter of the anatomy of interest based at least in part on the location of the obstruction relative to the anatomy of interest. The obstruction may obscure the at least a portion of the perimeter of the anatomy in the first image data.

Optionally, the first image data is an X-ray image.

Optionally, the at least one obstruction is at least one surgical instrument.

According to an aspect, a system for determining an attribute associated with anatomy of interest of a patient includes one or more processors, memory, and one or more programs stored in the memory for execution by the one or more processors and including instructions for: receiving first image data capturing the anatomy of interest of the patient and at least one obstruction obscuring at least a portion of the anatomy of interest of the patient; determining a location of the obstruction relative to the anatomy of interest within the first image data using at least one machine learning model; determining at least one attribute associated with the anatomy of interest based on the location of the obstruction relative to the anatomy of interest.

Optionally, the system further comprises instructions for generating a visual guidance associated with the anatomy of interest based on the determined at least one attribute and displaying the visual guidance.

Optionally, the visual guidance provides guidance for bone removal. The system may be configured to display the visual guidance intraoperatively for guiding a surgical procedure.

Optionally, determining the at least one attribute comprises identifying at least a portion of a perimeter of the anatomy of interest based at least in part on the location of the obstruction relative to the anatomy of interest. The obstruction may obscure the at least a portion of the perimeter of the anatomy in the first image data.

Optionally, the first image data is an X-ray image.

Optionally, the at least one obstruction is at least one surgical instrument.

According to an aspect, a method for compensating for an obstruction in imaging of anatomy of a patient includes receiving image data capturing anatomy of interest of the patient and at least one obstruction obscuring a portion of the anatomy of interest; detecting the at least one obstruction in the image data using at least one machine learning model; generating a data set from the image data in which at least a portion of the at least one obstruction is altered based on the anatomy of interest; determining at least one attribute associated with the anatomy of interest based on the data representation; generating a visual guidance associated with the anatomy of interest based on the determined at least one attribute; and displaying the visual guidance.

Optionally, the visual guidance provides guidance for bone removal.

Optionally, the visual guidance is displayed intraoperatively for guiding a surgical procedure.

Optionally, determining the at least one attribute comprises identifying at least a portion of a perimeter of the anatomy of interest based at least in part on the data set. The obstruction may obscure the at least a portion of the perimeter in the first image data.

Optionally, the image data is an X-ray image.

Optionally, generating the data set comprises using a first machine learning model to identify the obstruction and using a second machine learning model to generate the data set based on the identification of the obstruction by the first machine learning model.

Optionally, the visual guidance comprises a representation of the at least one obstruction overlaid on the representation of the at least a portion of the anatomy of interest.

Optionally, the at least one obstruction is at least one surgical instrument.

According to an aspect, a system for compensating for an obstruction in imaging of anatomy of a patient includes one or more processors, memory, and one or more programs stored in the memory for execution by the one or more processors and including instructions for: receiving image data capturing anatomy of interest of the patient and at least one obstruction obscuring a portion of the anatomy of interest; detecting the at least one obstruction in the image data using at least one machine learning model; generating a data set from the image data in which at least a portion of the at least one obstruction is altered based on the anatomy of interest; determining at least one attribute associated with the anatomy of interest based on the data representation; generating a visual guidance associated with the anatomy of interest based on the determined at least one attribute; and displaying the visual guidance.

Optionally, the visual guidance provides guidance for bone removal.

Optionally, the system is configured for displaying the visual guidance intraoperatively for guiding a surgical procedure.

Optionally, determining the at least one attribute comprises identifying at least a portion of a perimeter of the anatomy of interest based at least in part on the data set. The obstruction may obscure the at least a portion of the perimeter in the first image data.

Optionally, the image data is an X-ray image.

Optionally, generating the data set comprises using a first machine learning model to identify the obstruction and using a second machine learning model to generate the data set based on the identification of the obstruction by the first machine learning model.

Optionally, the visual guidance comprises a representation of the at least one obstruction overlaid on the representation of the at least a portion of the anatomy of interest.

Optionally, the at least one obstruction is at least one surgical instrument.

According to an aspect, a method for removing an obstruction from imaging of anatomy of a patient includes receiving image data capturing anatomy of interest of the patient and at least one obstruction obscuring a portion of the anatomy of interest; detecting the at least one obstruction in the image data; and generating second image data in which at least a portion of the at least one obstruction is replaced based on the anatomy of interest, the second image data including an outline of the at least one obstruction.

Optionally, the at least one obstruction is replaced by a representation of the portion of the anatomy of interest obscured by the at least one obstruction.

Optionally, the first image data comprises X-ray image data.

Optionally, the at least one obstruction is detected using a first machine learning model and the second image data is generated using a second machine learning model that is different than the first machine learning model. The first machine learning model may output a mask that indicates which pixels of the first image data correspond to the at least one obstruction. The second machine learning model may generate the representation based on the mask and the first image data. The mask may be enlarged prior to being provided as an input to the second machine learning model.

Optionally, the second image data is displayed intraoperatively.

Optionally, the method further includes determining at least one attribute associated with the anatomy of interest based on the second image data, generating a visual guidance associated with the anatomy of interest based on the determined at least one attribute, and adding the visual guidance to the second image data. The visual guidance may provide guidance for bone removal.

Optionally, the at least one obstruction includes an instrument or an implant.

According to an aspect, a system for removing an obstruction from imaging of anatomy of a patient includes one or more processors, memory, and one or more programs stored in the memory for execution by the one or more processors and including instructions for: receiving image data capturing anatomy of interest of the patient and at least one obstruction obscuring a portion of the anatomy of interest; detecting the at least one obstruction in the image data; and generating second image data in which at least a portion of the at least one obstruction is replaced based on the anatomy of interest, the second image data including an outline of the at least one obstruction.

Optionally, the at least one obstruction is replaced by a representation of the portion of the anatomy of interest obscured by the at least one obstruction.

Optionally, the first image data comprises X-ray image data.

Optionally, the at least one obstruction is detected using a first machine learning model and the second image data is generated using a second machine learning model that is different than the first machine learning model. Optionally, the first machine learning model outputs a mask that indicates which pixels of the first image data correspond to the at least one obstruction. Optionally, the second machine learning model generates the representation based on the mask and the first image data. Optionally, the mask is enlarged prior to being provided as an input to the second machine learning model.

Optionally, the system is configured to display the second image data intraoperatively.

Optionally, the system further includes instructions for: determining at least one attribute associated with the anatomy of interest based on the second image data, generating a visual guidance associated with the anatomy of interest based on the determined at least one attribute, and adding the visual guidance to the second image data. The visual guidance may provide guidance for bone removal.

Optionally, the at least one obstruction comprises an instrument or an implant.

According to an aspect, a non-transitory computer readable storage medium stores one or more programs, the one or more programs comprising instructions for execution by a computing system for performing any one of the above methods.

It will be appreciated that any of the variations, aspects, features and options described in view of the systems apply equally to the methods and vice versa. It will also be clear that any one or more of the above variations, aspects, features and options can be combined.

### BRIEF DESCRIPTION OF THE FIGURES

The invention will now be described, by way of example only, with reference to the accompanying drawings, in which:

FIG. 1 is a schematic view of an exemplary surgical suite;

FIG. 2 illustrates an exemplary method for removing an obstruction from imaging of anatomy of a patient;

FIG. 3A illustrates an example of first image data that includes an obstruction obscuring a portion of anatomy;

FIG. 3B is an example of second image data in which the obstruction of FIG. 3A has been removed and replaced with a representation of the obscured anatomy;

FIG. 3C illustrates an example of the display of a representation of the obstruction of FIG. 3A included in the second image data;

FIG. 3D illustrates an exemplary visual guidance that may be automatically generated by a computing system based on the removal and replacement of an obstruction in imaging;

FIG. 4 is a block diagram of an exemplary method for identifying at least one obstruction in an image and replacing the at least one obstruction with a representation of anatomy obscured by the at least one obstruction;

FIGS. 5A and 5B illustrate an exemplary mask created from an exemplary image by a segmentation machine learning model;

FIG. 6 is a block diagram of an exemplary method for training a machine learning model to identify obstructions in image data;

FIGS. 7A and 7B illustrate the created of artificial training data for training a machine learning model to identify obstructions in image data; and

FIG. 8 illustrates an example of a computing system.

### DETAILED DESCRIPTION

Reference will now be made in detail to implementations and examples of various aspects and variations of systems and methods described herein. Although several exemplary variations of the systems and methods are described herein, other variations of the systems and methods may include aspects of the systems and methods described herein combined in any suitable manner having combinations of all or some of the aspects described.

Systems and methods according to the principles described herein can automatically identify obstructions in medical imaging data. The obstructions can be removed and replaced based on the anatomy of interest, and/or analysis of the imaging data can be performed that takes into account the obstructions to enable or improve image-based analysis of the anatomy of interest. For example, the obstruction may be replaced with a representation of the anatomy obscured by the obstructions. Medical imaging often captures objects that obscure portions of anatomy of interest of a patient or that cause distortions in the imaging that obscure portions of anatomy of interest of the patient. The objects can be, for example, surgical instruments, objects worn by the patient, devices placed within the imaging field of view used for image calibration or measurement, and implants or other foreign objects within the body. These objects and/or the distortions caused by these objects (collectively referred to herein as obstructions) can obscure anatomy of interest, which can hinder a medical practitioner's ability to visualize the anatomy of interest and can prevent image analysis algorithms from accurately analyzing the anatomy of interest. The systems and methods described herein can automatically identify the obstructions in the medical imaging and replace them with representations of the anatomy of interest obscured by the obstructions. This can provide better visualizations of the anatomy of interest that can be provided to the medical practitioners and/or enable or improve image-based analysis of the anatomy of interest.

According to various aspects, the obstructions are identified and replaced using at least one machine learning model that is trained on training images that include the anatomy of interest. With this training, the at least one machine learning model “knows” what is likely obscured by an obstruction and can generate a realistic representation of the obscured anatomy. Optionally, a first machine learning model may be used for identifying an obstruction in an image and a second machine learning model may be used for replacing the obstruction with a representation of the anatomy of interest obscured by the obstruction. The first machine learning model may segment the imaging or a region of interest of the imaging, outputting a mask that indicates which pixels belong to the obstruction. The second machine learning model may take as an input the original imaging and the mask and may fill in the portions of the imaging associated with the obstruction with a representation of the anatomy that is obscured.

Systems and methods according to the principles described herein can be used for removing and replacing any type of obstructions from any type of imaging in support of any type of medical diagnostics or procedure by training machine learning models on suitable training data. For example, surgical instruments can be removed from X-ray images generated intraoperatively to enable a surgeon to have better visualization of the surgical site and/or to enable or improve image-based automated analysis for guiding the surgeon during the surgical procedure. Distractors, screws, cages, and other surgical implants can be removed from spine X-ray imaging to improve visualization and/or image-based analysis. Fixtures or other objects affixed to the body, such as for orienting a machine vision system, can be removed from X-ray imaging to improve visualization and/or image-based analysis. Pacemakers seen in chest X-rays can be removed to support diagnostics of diseases such as pneumonia, emphysema, pulmonary edema, and COVID-19. Jewelry, clothing buttons, and debris in pockets seen in diagnostic X-rays of clothed patients can be removed and replaced. Bullets, shrapnel, and metal implants can be removed from three-dimensional imaging slices (such as computed tomography (CT) slices) before 3D segmentation to reduce CT artifacts. Artifacts caused by metal in a surgical table or other object within the imaging field of view can be removed and replaced. In some variations, anatomy itself can be removed and replaced, such as where one bone is partially obscuring another. Optionally, the systems and methods may remove and replace anatomical features that are obscuring other anatomical features. For example, the systems and methods may remove and replace bone that is obscuring bone of interest or soft tissue of interest.

Optionally, a representation of an obstruction that has been removed and replaced may be included in the imaging to indicate to the user where the obstruction was and that the portions associated with the obstruction are artificial. Any suitable representation can be used, including an outline of the obstruction or a partially transparent representation of the obstruction.

The removal and replacement of obstructions from medical imaging, according to the principles described herein, can be used intraoperatively for guiding a surgeon during a surgical procedure. For example, an image may be generated during a surgical procedure, analyzed for the presence of obstructions, scrubbed of the obstructions, and displayed or otherwise used during the surgical procedure. Obstruction removal and replacement can be used pre-operatively for diagnosis or treatment and can be used post-operatively for assessing treatment success and/or recovery. Obstruction removal and replacement may be used for non-surgical applications, such as for diagnosis or in support of non-surgical treatments.

As noted above, the machine learning model(s) are trained on training images that include the anatomy of interest. Generally, the greater the amount of training data, the better the performance of the machine learning model. According to an aspect, the training data is augmented by generating artificial training imaging data in which obstructions from images are artificially added to images that do not have obstructions. The obstructions can be added in different locations and orientations to increase the amount of training images. The images with artificial obstructions and masks associated with the artificial obstructions may be used for training a machine learning model to identify the obstructions.

In the following description, it is to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

Certain aspects of the present disclosure include process steps and instructions described herein in the form of an algorithm. It should be noted that the process steps and instructions of the present disclosure could be embodied in software, firmware, or hardware and, when embodied in software, could be downloaded to reside on and be operated from different platforms used by a variety of operating systems. Unless specifically stated otherwise as apparent from the following discussion, it is appreciated that, throughout the description, discussions utilizing terms such as “processing,” “computing,” “calculating,” “determining,” “displaying,” “generating” or the like, refer to the action and processes of a computer system, or similar electronic computing device, that manipulates and transforms data represented as physical (electronic) quantities within the computer system memories or registers or other such information storage, transmission, or display devices.

The present disclosure in some examples also relates to a device for performing the operations herein. This device may be specially constructed for the required purposes, or it may comprise a general-purpose computer selectively activated or reconfigured by a computer program stored in the computer. Such a computer program may be stored in a non-transitory, computer readable storage medium, such as, but not limited to, any type of disk, including floppy disks, USB flash drives, external hard drives, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each coupled to a computer system bus. Furthermore, the computers referred to in the specification may include a single processor or may be architectures employing multiple processor designs for increased computing capability. Suitable processors include central processing units (CPUs), graphical processing units (GPUs), field programmable gate arrays (FPGAs), and ASICs.

The methods, devices, and systems described herein are not inherently related to any particular computer or other apparatus. Various general-purpose systems may also be used with programs in accordance with the teachings herein, or it may prove convenient to construct a more specialized apparatus to perform the required method steps. The required structure for a variety of these systems will appear from the description below. In addition, the present invention is not described with reference to any particular programming language. It will be appreciated that a variety of programming languages may be used to implement the teachings of the present invention as described herein.

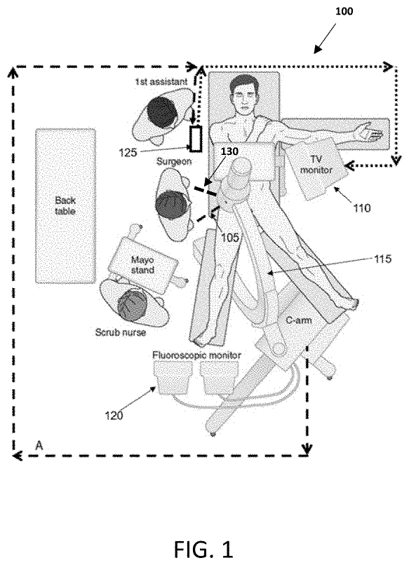

FIG. 1 illustrates an example of an arthroscopic surgical suite 100. In the arthroscopic surgical suite 100, the surgeon uses an arthroscope 105 and a display 110 to directly view an internal surgical site. The surgeon may use one or more instruments 130 in the internal surgical site. The surgeon may use a C-arm X-ray machine 115 and a display 120 to image the internal surgical site, such as to provide a larger view of aspects of interest of the surgical site than provided by the arthroscope 105. Imaging of the surgical site may be generated by the C-arm X-ray machine 115 while the one or more instruments 130 remain in the surgical site, and as a result, the imaging may capture one or more of the instruments 130. Often, an instrument obscures a region of interest of the surgical site. The surgical suite 100 can include a visual guidance system 125 that can identify the instrument in the imaging from the C-arm X-ray machine 115 and replace the obstruction in the imaging with a representation of at least a portion of the region of interest obscured by the instrument, according to the principles described herein. The resulting imaging can be displayed, such as via the visual guidance system 125, itself, and/or via display 110. The resulting imaging can additionally or alternatively be analyzed by visual guidance system 125 to determine one or more aspects of the anatomy that may have not have been possible to determine (or that may have been inaccurately determined) from the original imaging because of the instrument(s) obscuring the anatomy.

Visual guidance system 125 includes one or more processors, memory, and one or more programs stored in the memory for causing the visual guidance system to provide the functionality disclosed herein. Visual guidance system 125 can be configured as a tablet device with an integrated computer processor and user input/output functionality, e.g., a touchscreen. The visual guidance system 125 may be at least partially located in the sterile field, for example, the visual guidance system 125 may comprise a touchscreen tablet mounted to the surgical table or to a boom-type tablet support. The visual guidance system 125 may be covered by a sterile drape to maintain the surgeon's sterility as he or she operates the touchscreen tablet. Visual guidance system 125 may be configured as any other general purpose computer with appropriate programming and input/output functionality—for example, as a desktop or laptop computer with a keyboard, mouse, touchscreen display, heads-up display, gesture recognition device, voice activation feature, pupil reading device, etc. The visual guidance system 125 may be a distributed system in which at least a portion of its functionality is provided by a remote server, such as a cloud server. For example, a local portion of the visual guidance system 125 may provide imaging to a remote server, such as a cloud server, where image processing is conducted. Resulting imaging and/or analytical data may be returned from the remote server and may be displayed on a local display of the visual guidance system 125.

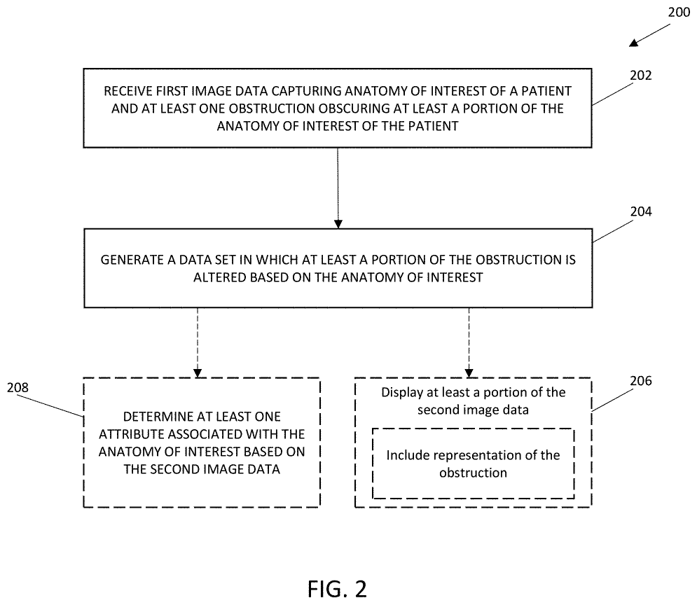

FIG. 2 illustrates a method 200 for compensating for an obstruction in imaging of anatomy of a patient, according to various aspects. Method 200 may be performed, for example, by visual guidance system 125 of FIG. 1. At step 202, first image data is received. The first image data captures anatomy of interest of a patient and at least one obstruction obscuring at least a portion of the anatomy of interest of the patient. The first image data can be generated by, for example, the C-arm X-ray machine 115 of FIG. 1. The first image data may have been generated by any suitable imaging modality, including, for example, X-ray imaging (including radiographic imaging and fluoroscopic imaging), visible light imaging, magnetic resonance imaging (MRI), computed tomography (CT) imaging, and ultrasound imaging. The first image data can be, for example, a single snapshot image, one or more video frames, or one or more slices of a three-dimensional imaging modality (for example, MRI or CT). The first image data may be received from an imaging system, such as C-arm X-ray machine 115, or may be received from a memory storing the imaging data which has previously been generated and stored. The first image data may be received by any suitable computing system, such as visual guidance system 125 of FIG. 1, another computing system within a medical room, a computing system in a medical facility, or a server system accessed via a client.

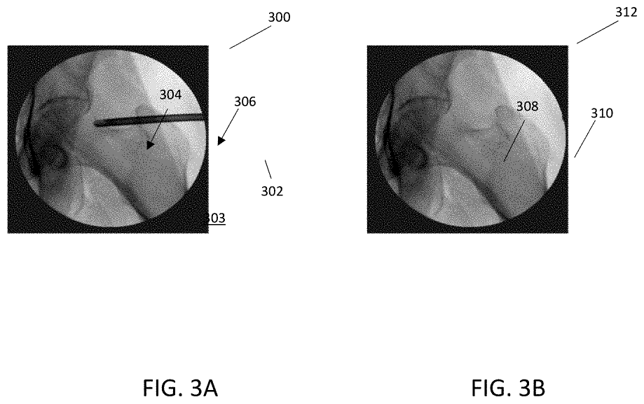

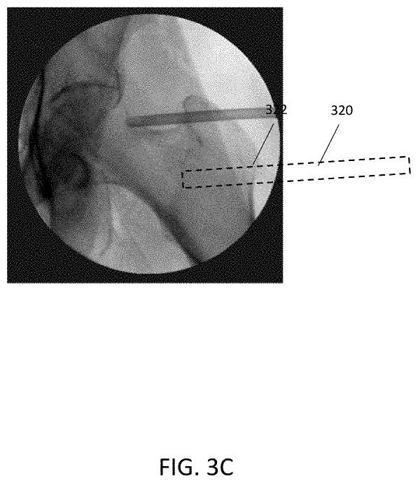

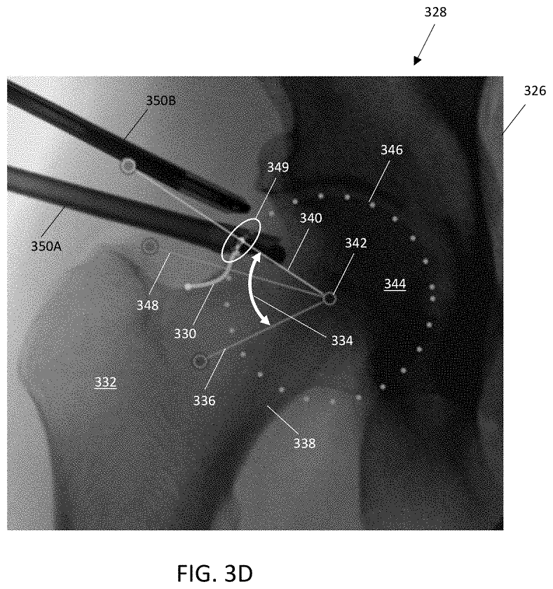

FIG. 3A illustrates an example of first image data that may be received at step 202. The first image data of FIG. 3A is an X-ray image 300 of a portion of a hip joint. Portions (304, 306) of the femur 303 of the hip joint are obscured in the image 300 by an obstruction 302 that was located in the field of view when the image 300 was generated. The obstruction 302 may be, for example, a surgical instrument, such as burrs and osteotomes, used during a surgical procedure on the hip joint.

At step 204, a data set is generated from the first image data that accounts for the obstruction (or obstructions). The data set can include an image, a series of images, video frames, and/or a volume, such as a DICOM data set, or any other simulation of a physical space, whether two or three dimensional. For example, the data set can be a second image in which at least a portion of the obstruction (or obstructions) has been altered based on the anatomy of interest. For example, at least a portion of the obstruction may be replaced by a representation of the anatomy obscured by the obstruction, or by a representation of other background or surrounding context within the data set. Altering of the at least one obstruction can include adding a degree of transparency to the at least one obstruction (for example, as an overlay) and displaying background or other context that is at least partially visible through the at least one obstruction (depending on the degree of transparency). Optionally, the altering of the obstruction in the data set can be done using at least one machine learning model. The at least one machine learning model may be trained to differentiate obstructions from anatomy in image data and may identify the obstruction in the first image data. The at least one machine learning model may remove the obstruction from the first image data and replace it with a representation of the anatomy that the obstruction may be obscuring.

According to various aspects, where the imaging data includes video or other time series of images, method 200 may be applied to each image frame using only the given image frame and not any previous image frames. In other words, the identification and/or replacement of obstructions according to method 200 does not use information from previous or later frames.

FIG. 3B is an example of second image data generated according to step 204. The image 312 in FIG. 3B was generated from the image 300 of FIG. 3A. In the image 312, the portions of the image 300 corresponding to the obstruction 302 have been replaced with representations 308, 310 of the portions 304, 306 of the femur obscured by the obstruction 302.

Returning to FIG. 2, method 200 may include an optional step 206 of displaying at least a portion of the second image data. For example, with reference to FIG. 3B, image 312 may be displayed. The second image data may be displayed during a surgical procedure, such as on display 110 of the surgical suite of FIG. 1, for guiding the surgical procedure. The second image data may be displayed prior to a medical procedure, such as for diagnosis and/or treatment planning. The second image data may be displayed after a medical procedure, such as for evaluating the medical procedure outcome and/or recovery of the patient.

Optionally, a representation of the obstruction in the image may be included in the display of the second image data. For example, a silhouette of the obstruction may be displayed in the location of the obstruction in the first image data, which could be done, for example, by blending together the first image data and the second image data. Other examples of representations of the obstruction are an outline of the obstruction overlaid on the second image data and altering an intensity of pixels associated with the removed obstruction. Including a representation of the obstruction in the display of the second image data may serve to inform the viewer that the portion of the anatomy in the region of the representation of the obstruction was artificially generated. FIG. 3C illustrates an example of the display of a representation of the obstruction 302, which is overlaid on the image generated in step 204. The representation includes a semi-transparent reproduction 320 and an outline 322 of the obstruction. Optionally, a user can control the display of the representation of the obstruction, which could include toggling display of the representation and/or adjusting a transparency of the representation of the obstruction from completely transparent to completely opaque.

Method 200 may include the optional step 208 of determining, by the computing system, at least one attribute associated with the anatomy of interest based on the data set generated at step 204. By altering the obstruction based on the anatomy of interest obscured by the obstruction, the determination of one or more attributes of the anatomy in the image may be improved or enabled. For example, in some variations, an image analysis algorithm determines an attribute of the anatomy at least partially relying upon aspects of the anatomy that would otherwise be obscured by the obstruction, and the algorithm may not have been able to determine the attributes (or the determination of the attributes may have been unreliable) due to insufficient information resulting from the obstruction obscuring portions of the anatomy. Thus, by altering the obstruction in generating the data set (for example, via removal of the obstruction and replacement with a representation of the obscured anatomy) a sufficient approximation of the missing information may be provided in the data set to enable the algorithm to determine the attributes or generate more reliable estimates of the attributes. Step 208 can be done with or without the displaying of a second image in step 206.

An example of the determination of at least one attribute associated with the anatomy of interest based on image data in which an obstruction has been removed and replaced with a representation of obscured anatomy, according to step 208, is described below with reference to FIG. 3D.

FIG. 3D illustrates an exemplary visual guidance 328 that may be automatically generated by a computing system, such as visual guidance system 125, for guiding a femoral debridement procedure for treatment of cam-type femoroacetabular impingement. With cam-type femoroacetabular impingement, irregularities in the geometry of the femur can lead to impingement between the femur and the rim of the acetabular cup. Treatment for cam-type femoroacetabular impingement typically involves debriding the femoral neck and/or head, using instruments such as burrs and osteotomes, to remove the bony deformities causing the impingement. The visual guidance 328 of FIG. 3D includes an X-ray image 326 of a hip joint and a resection curve 330 overlaid on the X-ray image 326 for indicating to a surgeon where to debride the femoral neck and/or head to treat the cam-type femoroacetabular impingement.

The resection curve 330 may be automatically determined from a number of attributes of the femur 332. A common anatomical measurement used in diagnosing cam-type femoroacetabular impingement (FAI) is the Alpha Angle 334. The Alpha Angle is defined as the angle between a line 336 extending along the mid-line of the femoral neck 338 and a line 340 that originates at the center 342 of the femoral head 344 and passes through the location where the bone first extends outside a circle 346 set at the perimeter of the femoral head 344 (the start of the cam pathology). A healthy hip typically has an Alpha Angle of anywhere from less than approximately 42 degrees to approximately 50 degrees. Thus, a patient with an Alpha Angle of greater than approximately 50 degrees may be a candidate for FAI surgery. The resection curve 330 may guide a surgeon in the removal of bone to reduce the Alpha Angle 334 to a desired target 348.

The image 326 includes two instruments 350A, 350B, one of which is obscuring a portion of the femoral head 344. This obscuring by instrument 350A may adversely affect the ability to determine the attributes of the femur 332 used for generating the resection curve 330. In the illustrated example, instrument 350A is obscuring the location 349 where the bone first extends outside a circle 346, making an accurate determination of the Alpha Angle 334 difficult or impossible using conventional means. Additionally, the perimeter of the femoral head 344 may not be accurately determinable due to the instrument 350A obscuring a portion of the perimeter.

According to step 208, the instrument 350A can be removed and replaced with a representation of the portion of the femur 332 that is obscured by the instrument 350A. With the missing information replaced, the attributes of the anatomy in the image (e.g., one or more of the circle 346 set at the perimeter of the femoral head 344, the center 342 of the femoral head 344, the mid-line 336 of the femoral neck 338, the location where the bone first extends outside a circle 346, the Alpha Angle 334, etc.) can be determined. For example, the perimeter of the femoral head 344 may be more accurately determined and the location where the bone first extends outside a circle 346 placed on the perimeter of the femoral head 344 may be determined. In this way, the system can more accurately determine the attributes of the femur 332 needed to generate the resection curve 330. After determining the attributes based on the obstruction-free image data, a visual guidance can be added to the obstruction-free image. For example, the resection curve 330 and/or the attributes of the anatomy may be overlaid on the obstruction-free image, with or without a representation of the obstruction. Thus, method 200 can not only enable visualization of the portions of anatomy obscured by obstructions in an image, but can also enable or improve automatic analysis of the anatomy in the image.

In some variations, the attribute of the anatomy of interest is determined without generating a data set in which the obstruction is altered. The determination of the attribute is determined by taking into account the obstruction but without first creating any alteration of the obstruction (i.e., without first creating a second image in which the obstruction has been altered). The obstruction in the first image data may be detected and its location relative to the anatomy of interest in the first image data may be determined. This can be done, for example, using one or more machine learning models that can identify the obstruction(s) and the anatomy of interest. With the knowledge of the location of the obstruction relative to the anatomy of interest, a determination of the attribute(s) can be done that takes into account the location of the obstruction relative to the anatomy of interest. For example, pixel data associated with an intersection between the obstruction and the anatomy of interest (where the obstruction overlaps with the anatomy of interest) may be ignored during determination of the attribute. Using FIG. 3D to illustrate, one or more of the circle 346, the center 342 of the femoral head 344, and the mid-line 336 of the femoral neck 338 can be determined on one or more sets of pixel data in which the pixel data associated with the obstruction 350A have been explicitly excluded. In some examples, after determining the attribute(s) based on the location of the obstruction relative to the anatomy of interest, a visual guidance can be generated and displayed to a user. For example, the resection curve 330 and/or the attributes of the anatomy may be overlaid on the first image, as illustrated in FIG. 3D.

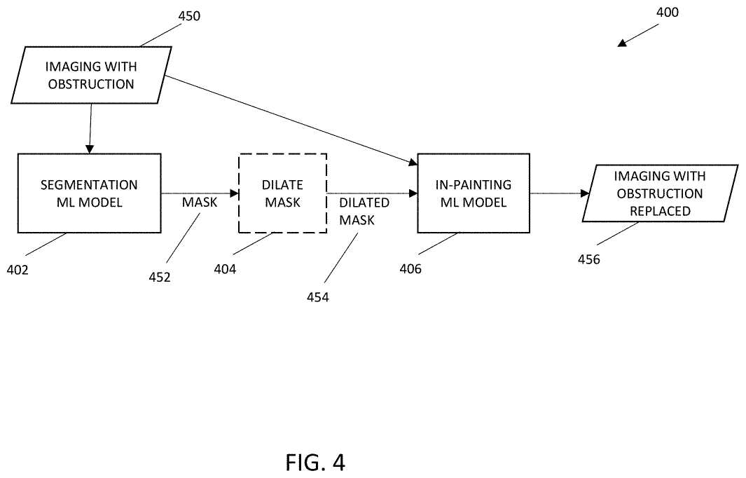

FIG. 4 is a block diagram of an exemplary method 400 for identifying at least one obstruction in an image and replacing the at least one obstruction with a representation of anatomy obscured by the at least one obstruction. Method 400 could be used, for example, for step 204 of method 200 of FIG. 2. As discussed further below, method 400 includes using a first machine learning model to identify the at least one obstruction in the image and a second machine learning model to fill in the portions of the image corresponding to the at least one obstruction to provide a representation of the portions of the anatomy obscured by the at least one obstruction.

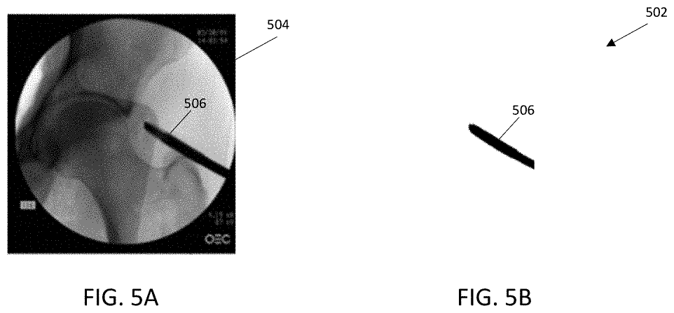

At step 402, a segmentation machine learning model is used to identify at least one obstruction in the image data 450. The machine learning model segments the image data 450 into pixels that are associated with the obstruction and pixels that are not. The first machine learning model outputs a mask 452 corresponding to the pixels associated with the obstruction. FIGS. 5A and 5B illustrate a mask 502 created from an exemplary image 504 by a segmentation machine learning model according to step 402. The mask 502 corresponds to an obstruction 506 obscuring a portion of the anatomy captured in the image 504.

Optionally, the segmentation machine learning model may search only a region of interest of image data for obstructions. For example, for image 300 of FIG. 3A, the segmentation machine learning model may search only the field of view portion of the image 300 (the circular portion). A different machine learning model or other image analysis technique may be used to identify the region of interest of the image data and may provide this information to the segmentation machine learning model.

The machine learning model used in step 402 may be a convolutional neural network (CNN) configured for biomedical segmentation. The convolutional neural network may be a fully convolutional network. Examples of suitable neural networks include U-Net, Gated Shape CNN (Gated-SCNN), DeepLab, and Mask regional CNN (Mask RCNN). Optionally, a neural network may be used to identify obstructions in the image data and an edge-based segmentation method may be used to delineate the obstruction in the image.

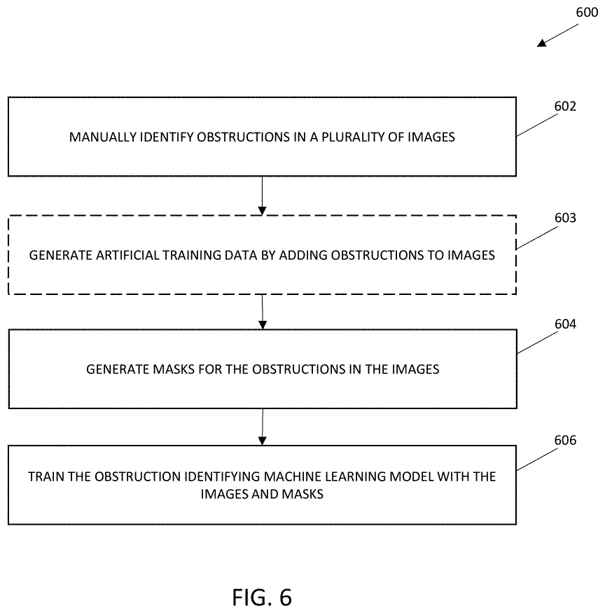

According to an aspect, the segmentation machine learning model used in step 402 is trained using a supervised learning technique. FIG. 6 is a block diagram of an exemplary method 600 for training a machine learning model to identify obstructions in image data. At step 602, obstructions are identified in a plurality of training images or other suitable training imaging data, and the obstructions are outlined or otherwise delimited. This step may be performed manually. The outlined region(s) in each training image are then converted to masks in step 604. At step 606, the machine learning model is trained on the masks and training images.

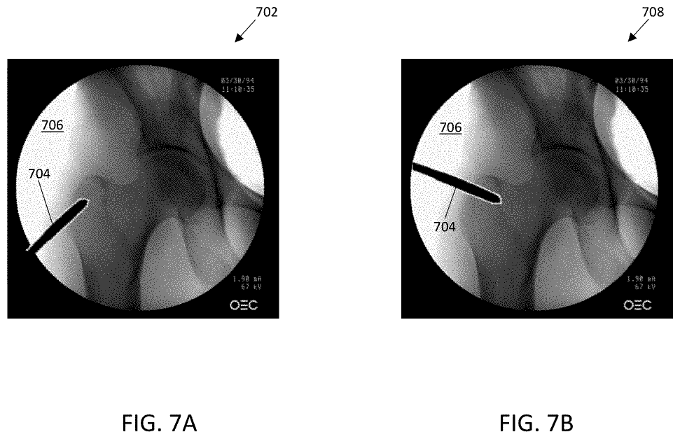

Since the performance of a machine learning model is generally improved by increasing the amount of training data, method 600 can include an optional step 603 in which artificial training images are generated to increase the amount of training data. Step 603 can include cutting obstructions out of the images that have them and adding the obstructions to images that do not have obstructions. This may be done manually. A mask is created that indicates where the obstruction is positioned in an image. The obstructions can be rotated, translated, changed in size, used in part, placed on different portions of anatomy, placed multiple times in an image, or otherwise used in different ways to increase the amount and variability of training data. FIGS. 7A and 7B illustrate an example of the creation of artificial training data according to step 603. A first training image 702, shown in FIG. 7A, was generated by adding an obstruction 704 (extracted from, for example, the image 504 of FIG. 5A) in a suitable orientation and position to an obstruction-free image 706. A second training image 710, shown in FIG. 7B, was generated by adding the same obstruction 704 in a different orientation and/or position in the same obstruction-free image 706.

Returning to FIG. 4, the segmentation performed in step 402 may not accurately identify all pixels associated with an obstruction, and therefore, the mask 452 may not encompass all pixels associated with the obstruction. Missed pixels could cause the replacement of the obstruction to look less realistic. To reduce the likelihood of missed pixels, method 400 can include an optional step 404 of adjusting a size of the mask 452. The size of the mask 452 could be adjusted by dilating the mask 452 to increase the likelihood that all pixels associated with the obstruction are encompassed by the mask. Dilation of the mask 452 could also be used to encompass any imaging-based distortion associated with the obstruction, such as a halo or ghosting effect that often occurs around instruments captured by some X-ray imaging systems.

Method 400 continues with step 404 in which an in-painting machine learning model fills in the portions of the image data 450 with a representation of the anatomy obscured by the obstruction. The mask 452 or dilated mask 454 and the original image data 450 are inputs to the second machine learning model. The in-painting machine learning model has a degree of “understanding” of how anatomy should look, and therefore, can replace obstructions with image segments that look like real anatomy. The in-painting machine learning model does not merely blend or extrapolate from surrounding image regions. For example, if an obstruction were to obscure a distinct portion of anatomy entirely, the in-painting machine learning model may be trained to know that the distinct portion of anatomy is normally in that location and will add a representation that looks realistic. In contrast, traditional methods try to conform the region to surrounding regions, which often does not look realistic. The in-painting machine learning model can be a partial convolutional neural network (Pconv). Other suitable in-painting neural networks include a Generative Multi-column Convolutional Neural Networks (GMCCNN) and a convolutional auto encoder.

In some variations, the in-painting machine learning model can be trained using an unsupervised training technique. A set of training images that do not have tools may be used. Masks of a number of suitable shapes may be generated for the training images. The shapes may be chosen for tailoring to a particular application. For example, where a given application is likely to include surgical instruments that obscure portions of tissue in images, shapes that are similar to the surgical instruments, such as thick lines and/or ovals, may be used. The pixels encompassed by the masks are set to zero and the machine learning model is trained to set the pixel values to produce a realistic representation of what is likely to have been there. The machine learning model is not being trained to re-create the original image (e.g., the original image is not used as ground truth) but to fill-in the masked area in a realistic way.

In some variations, the in-painting machine learning model can be a diffusion-based model. The diffusion-based model decompose the image generation process into a sequential application of denoising autoencoders. The diffusion-based model is trained by first applying noise iteratively to a set of training images and then recovering the data by reversing the noising process using denoising autoencoders. The trained diffusion-based model can perform in-painting tasks by sequentially applying the denoising autoencoders to the obstruction identified in step 402 to generate a new representation from the noise pattern that is coherent with the rest of the image. In some variations, the diffusion-based model can be a latent diffusion model, including, but not limited to, Stable Diffusion. The latent diffusion model can apply the diffusion process in latent space instead of pixel space, thereby enhancing the computational efficiency of the process as compared to other diffusion models, such as pixel-based diffusion models.

The output of step 404 is image data 456 that includes a representation of anatomy obscured by the obstruction in place of the obstruction. Image data 456 can then be displayed and/or used for analysis, for example, as described above in step 206 and/or step 208 of method 200.

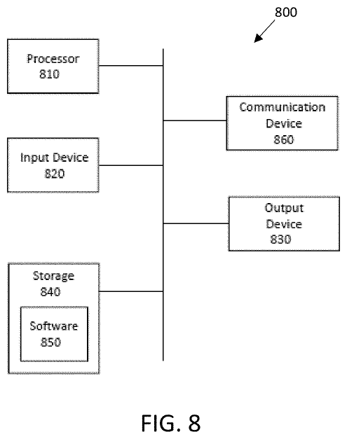

FIG. 8 illustrates an example of a computing system, in accordance with some examples, that can be used for performing any of the methods described herein, including method 200 of FIG. 2, method 400 of FIG. 4, and method 600 of FIG. 6, and can be used for any of the systems described herein, including visual guidance system 125 of FIG. 1. System 800 can be a computer connected to a network, which can be, for example, an operating room network or a hospital network. System 800 can be a client computer or a server. As shown in FIG. 8, system 800 can be any suitable type of microprocessor-based system, such as a personal computer, workstation, server, or handheld computing device (portable electronic device) such as a phone or tablet. The system can include, for example, one or more of processor 810, input device 820, output device 830, storage 840, and communication device 860. Input device 820 and output device 830 can generally correspond to those described above and can either be connectable or integrated with the computer.

Input device 820 can be any suitable device that provides input, such as a touch screen, keyboard or keypad, mouse, gesture recognition component of a virtual/augmented reality system, or voice-recognition device. Output device 830 can be or include any suitable device that provides output, such as a touch screen, haptics device, virtual/augmented reality display, or speaker.

Storage 840 can be any suitable device that provides storage, such as an electrical, magnetic, or optical memory including a RAM, cache, hard drive, removable storage disk, or other non-transitory computer readable medium. Communication device 860 can include any suitable device capable of transmitting and receiving signals over a network, such as a network interface chip or device. The components of the computer can be connected in any suitable manner, such as via a physical bus or wirelessly.

Software 850, which can be stored in storage 840 and executed by processor 810, can include, for example, the programming that embodies the functionality of the present disclosure (e.g., as embodied in the devices as described above). For example, software 850 can include one or more programs for performing one or more of the steps of method 200, method 400, and/or method 600.

Software 850 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 840, that can contain or store programming for use by or in connection with an instruction execution system, apparatus, or device.

Software 850 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic, or infrared wired or wireless propagation medium.

System 800 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

System 800 can implement any operating system suitable for operating on the network. Software 850 can be written in any suitable programming language, such as C, C++, Java, or Python. In various examples, application software embodying the functionality of the present disclosure can be deployed in different configurations, such as in a client/server arrangement or through a Web browser as a Web-based application or Web service, for example.

The foregoing description, for the purpose of explanation, has been described with reference to specific examples. However, the illustrative discussions above are not intended to be exhaustive or to limit the invention to the precise forms disclosed. Many modifications and variations are possible in view of the above teachings. The examples were chosen and described in order to best explain the principles of the techniques and their practical applications. Others skilled in the art are thereby enabled to best utilize the techniques and various examples with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

1. A method for determining an attribute associated with anatomy of interest of a patient comprising:
receiving first image data capturing the anatomy of interest of the patient and at least one obstruction obscuring at least a portion of the anatomy of interest of the patient;
generating, using at least one machine learning model, second image data in which at least a portion of the obstruction is replaced; and
determining at least one attribute associated with the anatomy of interest based on the second image data.

2. The method of claim 1, further comprising generating a visual guidance associated with the anatomy of interest based on the determined at least one attribute and adding the visual guidance to the second image data.

3. The method of claim 1, wherein the visual guidance provides guidance for bone removal.

4. The method of claim 1, wherein the second image data is displayed intraoperatively for guiding a surgical procedure.

5. The method of claim 1, wherein determining the at least one attribute comprises identifying at least a portion of a perimeter of the anatomy of interest based at least in part on the representation of the at least a portion of the anatomy of interest obscured by the obstruction.

6. The method of claim 5, wherein the obstruction obscures the at least a portion of the perimeter in the first image data.

7. The method of claim 1, wherein the first image data is an X-ray image.

8. The method of claim 1, wherein generating the second image data comprises using a first machine learning model to identify the obstruction and using a second machine learning model to generate the second image data based on the identification of the obstruction by the first machine learning model.

9. The method of claim 1, further comprising displaying the second image data with a representation of the at least one obstruction overlaid on the representation of the at least a portion of the anatomy of interest.

10. The method of claim 1, wherein the at least one obstruction is at least one surgical instrument.

11. The method of claim 1, wherein the at least one machine learning model comprises a diffusion-based machine learning model.

12. A system for determining an attribute associated with anatomy of interest of a patient, the system comprising one or more processors, memory, and one or more programs stored in the memory for execution by the one or more processors and including instructions for:
receiving first image data capturing the anatomy of interest of the patient and at least one obstruction obscuring at least a portion of the anatomy of interest of the patient;
generating, using at least one machine learning model, second image data in which at least a portion of the obstruction is replaced; and
determining at least one attribute associated with the anatomy of interest based on the second image data.

13. The system of claim 12, wherein the one or more programs include further instructions for generating a visual guidance associated with the anatomy of interest based on the determined at least one attribute and adding the visual guidance to the second image data.

14. The system of claim 12, wherein the visual guidance provides guidance for bone removal.

15. The system of claim 14, wherein the second image data is displayed intraoperatively for guiding a surgical procedure.

16. The system of claim 12, wherein determining the at least one attribute comprises identifying at least a portion of a perimeter of the anatomy of interest based at least in part on the representation of the at least a portion of the anatomy of interest obscured by the obstruction.

17. The system of claim 16, wherein the obstruction obscures the at least a portion of the perimeter in the first image data.

18. The system of claim 12, wherein the first image data is an X-ray image.

19. The system of claim 12, wherein generating the second image data comprises using a first machine learning model to identify the obstruction and using a second machine learning model to generate the second image data based on the identification of the obstruction by the first machine learning model.

20. The system of claim 12, wherein the one or more programs include further instructions for displaying the second image data with a representation of the at least one obstruction overlaid on the representation of the at least a portion of the anatomy of interest.

21. The system of claim 12, wherein the at least one obstruction is at least one surgical instrument.

22. A method for determining an attribute associated with anatomy of interest of a patient comprising:
receiving first image data capturing the anatomy of interest of the patient and at least one obstruction obscuring at least a portion of the anatomy of interest of the patient;
determining a location of the obstruction relative to the anatomy of interest within the first image data using at least one machine learning model; and
determining at least one attribute associated with the anatomy of interest based on the location of the obstruction relative to the anatomy of interest.

23. The method of claim 22, further comprising generating a visual guidance associated with the anatomy of interest based on the determined at least one attribute and displaying the visual guidance.

24. The method of claim 23, wherein the visual guidance provides guidance for bone removal.

25. The method of claim 21, wherein determining the at least one attribute comprises identifying at least a portion of a perimeter of the anatomy of interest based at least in part on the location of the obstruction relative to the anatomy of interest.

26. The method of claim 25, wherein the obstruction obscures the at least a portion of the perimeter of the anatomy in the first image data.

27. A system for determining an attribute associated with anatomy of interest of a patient, the system comprising one or more processors, memory, and one or more programs stored in the memory for execution by the one or more processors and including instructions for:
receiving first image data capturing the anatomy of interest of the patient and at least one obstruction obscuring at least a portion of the anatomy of interest of the patient;
determining a location of the obstruction relative to the anatomy of interest within the first image data using at least one machine learning model; and
determining at least one attribute associated with the anatomy of interest based on the location of the obstruction relative to the anatomy of interest.

28. A method for compensating for an obstruction in imaging of anatomy of a patient comprising:
receiving image data capturing anatomy of interest of the patient and at least one obstruction obscuring a portion of the anatomy of interest;
detecting the at least one obstruction in the image data using at least one machine learning model;
generating a data set from the image data in which at least a portion of the at least one obstruction is altered based on the anatomy of interest;
determining at least one attribute associated with the anatomy of interest based on the data representation;
generating a visual guidance associated with the anatomy of interest based on the determined at least one attribute; and
displaying the visual guidance.

29. The method of claim 28, wherein the visual guidance provides guidance for bone removal.

30. The method of claim 28, wherein determining the at least one attribute comprises identifying at least a portion of a perimeter of the anatomy of interest based at least in part on the data set.

31. The method of claim 30, wherein the obstruction obscures the at least a portion of the perimeter in the first image data.

32. The method of claim 28, wherein generating the data set comprises using a first machine learning model to identify the obstruction and using a second machine learning model to generate the data set based on the identification of the obstruction by the first machine learning model.

33. The method of claim 28, wherein the visual guidance comprises a representation of the at least one obstruction overlaid on the representation of the at least a portion of the anatomy of interest.

34. A system for compensating for an obstruction in imaging of anatomy of a patient, the system comprising one or more processors, memory, and one or more programs stored in the memory for execution by the one or more processors and including instructions for:
receiving image data capturing anatomy of interest of the patient and at least one obstruction obscuring a portion of the anatomy of interest;
detecting the at least one obstruction in the image data using at least one machine learning model;
generating a data set from the image data in which at least a portion of the at least one obstruction is altered based on the anatomy of interest;
determining at least one attribute associated with the anatomy of interest based on the data representation;
generating a visual guidance associated with the anatomy of interest based on the determined at least one attribute; and
displaying the visual guidance.
